(19) United States
(12) Patent Application Publication
(10) Pub. No.: US 2007/0009935 A1
(43) Pub. Date: Jan. 11, 2007

(54) Arrangements, systems and methods capable of providing spectral-domain optical coherence reflectometry for a sensitive detection of chemical and biological sample

(75) Inventors: Chulmin Joo, Cambridge, MA (US); Johannes F. De Boer, Somerville, MA (US)

Correspondence Address: DORSEY & WHITNEY LLP;INTELLECTUAL PROPERTY DEPARTMENT, 250 PARK AVENUE, NEW YORK, NY, 10177, US

(73) Assignee: The General Hospital Corporation

(21) Appl. No.: 11/435,228

(22) Filed: May 15, 2006

Related U.S. Application Data
(60) Provisional application No. 60/680,947, filed on May 13, 2005.

(51) Int. Cl. C12Q 1/68; C12M 3/00

(52) U.S. Cl. 435/6; 435/287.2

(57) ABSTRACT

Systems, arrangements and methods for a molecular recognition are provided. For example, a particular radiation having wavelength that varies over time and/or a spectral width that is greater than 10 nm can be provided. For example, at least one first electro-magnetic radiation can be provided to at least one sample, and at least one second electro-magnetic radiation may be provided to a reference, with both the first and second electro-magnetic radiations being part of the particular radiation. Further, the interference between a third electro-magnetic radiation (associated with the first electro-magnetic radiation) and a fourth electro-magnetic radiation (associated with the second electro-magnetic radiation) can be detected. A change in a thickness of at least one portion of the sample based on the interference can be determined.

### CROSS-REFERENCE TO RELATED APPLICATION(S)

This application is based upon and claims the benefit of priority from U.S. Patent Application Ser. No. 60/680,947, filed May 13, 2005, the entire disclosure of which is incorporated herein by reference.

### STATEMENT REGARDING FEDERALLY SPONSORED RESEARCH

The invention was made with the U.S. Government support under Contract No. RO1 EY014975 and RO1RR019768 awarded by the National Institute of Health, and Contract No. F49620-021-1-0014 awarded by the Department of Defense. Thus, the U.S. Government has certain rights in the invention.

### FIELD OF THE INVENTION

The present invention relates to methods and apparatus for a molecular recognition. More particularly, the present invention relates to detection arrangements, systems and methods for a molecular binding on a sensing surface and the presence of molecules in channels.

### BACKGROUND OF THE INVENTION

Real-time detection of minute traces of molecules (e.g., pesticides, viruses, and organic toxins) is important in various applications such as medical diagnostics, environmental monitoring, and homeland security. For example, there is a need for providing a highly sensitive detection methods of viruses, as well as processes that provide an early detection of chemicals and pathogens (e.g., explosives, anthrax) which could trigger a corrective action. Such methods may be important in a broad range of, e.g., medical and environmental applications and bio-defense.

Such exemplary detection has been conducted by fluorescent (as described in D. W. Pierce et al., “Imaging individual green fluorescent proteins,”. Nature, 1997, Vol. 388, pp. 338 et seq.) and using certain radioactive methods. Even though these label-based techniques could potentially achieve single molecular level detection, an additional specimen preparation is needed to be performed therefor, which is costly in time and may affect the molecules of interest.

Label-free detection techniques such as surface plasmon resonance (SPR) sensors (as described in J. Homola et al., “Surface plasmon resonance sensors: review,” Sensors and Actuators B, 1999, Vol. 54, pp. 3-15) and quartz crystal microbalances (QCM) arrangements (as described in G. Kleefisch et al., “Quartz microbalance sensor for the detection of Acrylamide,” Sensors, 2004, Vol. 4, pp. 136-146) provide an indication of a physical absorption of molecules on a sensor surface. The SPR sensor generally exploits the change of the SPR angle due to the alteration of refractive index at a metal-dielectric interface upon the protein absorption. However, this sensor may review a large amount of molecules, since its lateral resolution may not be reduced without loss of sensitivity (as described in C. Berger et al., “Resolution in surface plasmon microscopy,” REVIEW OF SCIENTIFIC INSTRUMENTS, 1994, Vol. 65, pp. 2829-2836). QCM techniques also utilize the shift of resonance frequency due to the effective mass increase upon the protein binding. In addition to the needed large amount of molecules, the QCM detection method needs to operate in a dry environment, preferably in a vacuum, because the damping in aqueous environment likely deteriorates the sensitivity.

Several methods based on micro-fabrication techniques have been (as provided in P. Burg et al, “Suspended microchannel resonators for biomolecular detection,” Applied Physics Letters, 2003, Vol. 83(13), pp. 2698-2700; and W. U. Wang et al., “Label-free detection of small-molecule-protein interactions by using nanowire nanosensors,” PROCEEDINGS OF THE NATIONAL ACADEMY OF SCIENCES OF THE UNITED STATES OF AMERICA, 2005. 102: p. 3208-3212), attempted to address the above-described deficiencies. Such methods could potentially achieve sensitive detection for label-free species, but the fabrication techniques (e.g., e-beam lithography, electron beam evaporation, and chemical vapor deposition) are complicated and expensive, and the sensing units that use such techniques are likely directly coupled to micro-fluidic devices, limiting their utility for various diagnostic applications.

A spectral domain optical coherence reflectometry (SD-OCR) technique is an optical ranging procedure which is capable of measuring depth-resolved phase information with a sub-nanometer thickness sensitivity. For example, a thickness change can be an optical thickness change, a refractive index change, and/or a physical thickness change. Detailed descriptions on SD-OCR and demonstration of sub-nanometer sensitivity are provided in International Patent Application PCT/US03/02349 and described in C. Joo et al., “Spectral-domain optical coherence phase microscopy for quantitative phase-contrast imaging,” Optics Letters, 2005, Vol. 30, pp. 2131-2133; and B. C. Nassif et al., “In vivo human retinal imaging by ultrahigh-speed spectral domain optical coherence tomography,” Optics Letters, 2004, Vol. 29, pp. 480-482.

### OBJECTS AND SUMMARY OF THE INVENTION

One of the objects of the present invention is to overcome certain deficiencies and shortcomings of the prior art systems (including those described herein above), and implement exemplary SD-OCR techniques as shall be described in further detail below. This can be done by implementing arrangements, systems and methods which utilize SD-OCR techniques (e.g., SD-OCR arrangements, systems and methods). Another object of the present invention is to utilize systems, arrangements and methods and apply SD-OCR techniques to obtain a highly sensitive detection of label-free chemical and biological species (e.g., anatomical samples).

For example, exemplary embodiments of the system, arrangement and method according to the present invention can be provided for label-free chemical and biological species. The exemplary embodiments can utilize a coherence gating of low-coherence interferometry to identify the interference signal of interest, and measures the phase alteration of that signal for molecular absorption/removal at a surface or concentration measurement in the channels. For molecular binding on a sensing surface, these exemplary embodiments can permit an examination of molecular interactions on a micron-sized area, and thus can be extended to monitoring a large number of activated sites in parallel on a two-dimensional surface in disposable arrays, and can be adapted for the detection of new chemical and biological species by including an active binding site into the micro arrays.

Therefore, systems, arrangements and methods for a molecular (e.g., for a molecular binding on a sensing surface and the presence of molecules in channels) are provided. For example, a particular radiation having wavelength that varies over time and/or a spectral width that is greater than 10 nm can be provided. For example, at least one first electro-magnetic radiation can be provided to at least one sample, and at least one second electro-magnetic radiation may be provided to a reference, with both the first and second electro-magnetic radiations being part of the particular radiation. Further, the interference between a third electro-magnetic radiation (associated with the first electro-magnetic radiation) and a fourth electro-magnetic radiation (associated with the second electro-magnetic radiation) can be detected. A change in a thickness of at least one portion of the sample based on the interference can be determined.

According to another exemplary embodiment of the present invention, the first and second radiations can share a common path. The sample can include a plurality of samples, and the change in the thickness of the at least one portion of each of the samples may be determined simultaneously. The change in the thickness of the at least one portion of the at least one samples may be determined simultaneously at different locations along and/or perpendicular to a beam path of the first electro-magnetic radiation. The change in the thickness may also be determined simultaneously along different locations along a beam path of the first electro-magnetic radiation. The first electro-magnetic radiation may be scanned over a surface of the sample at a plurality of locations thereon.

According to still another exemplary embodiment of the present invention, the portion of the sample may be coated with particular molecules that are designed to associate with or dissociate from to further molecules. The change of the thickness may be associated with an association or a dissociation of the particular molecules. The particular molecules may have an affinity to bind to the further molecules that are different from the particular molecules. The portion may include a plurality of portions. For example, a first set of the particular molecules may have an affinity to bind to a first portion of the portions, and a second set of the particular molecules can have an affinity to bind to a second portion of the of portions. The first and second sets may be different from one another.

In a further exemplary embodiment of the present invention, the sample can have multiple layers therein and/or may be disposable. The sample can be a micro-fluidic arrangement. The change of the thickness of the portion of the sample can be an optical thickess change and/or a physical thickness change and/or a refractive index change. The thickness change can be associated with a concentration of molecules of on and/or in the portion of the sample. The thickness can change as a function of wavelength that is associated with types of molecules of on and/or in the portion of the sample. The first electro-magnetic radiation may have a cross-section of a beam on and/or in the portion of the sample has a size that can be can be as small as a diffraction-limited size (e.g., 10 μm). The thickness can be determined by (i) transforming the interference into first data which is in a complex format, (ii) determining an absolute value associated with the first data to generate second data, (iii) identifying particular locations of the portion as a function of the second data, (iv) determining a phase associated with the first data to generate third data, and (v) associating the change of the thickness with the third data. Further, the interference may be Fourier transformed to generate the first data.

These and other objects, features and advantages of the present invention will become apparent upon reading the following detailed description of embodiments of the invention, when taken in conjunction with the appended claims.

### BRIEF DESCRIPTION OF THE DRAWINGS

Further objects, features and advantages of the invention will become apparent from the following detailed description taken in conjunction with the accompanying figures showing illustrative embodiments of the invention, in which:

FIG. 1 is a diagram of an exemplary embodiment of an SD-OCR biosensing arrangement in accordance with the present invention;

FIG. 2a is diagram of an exemplary usage of the exemplary arrangement of FIG. 1 for a measurement of a molecular interaction at a particular point in time in accordance with the present invention;

FIG. 2b is diagram of the exemplary usage of the exemplary arrangement of FIG. 1 for the measurement of the molecular interaction at a subsequent point in time in accordance with the present invention;

FIG. 2c is diagram of the exemplary usage of the exemplary arrangement of FIG. 1 for the measurement of the molecular interaction at a still subsequent point in time in accordance with the present invention;

FIG. 3 is a diagram of the exemplary embodiment of the SD-OCR arrangement which is illustrated as performing a SD-OCR depth-resolved measurement of the molecular interaction;

FIG. 4 is an exemplary operational measurement in accordance with an exemplary embodiment of the present invention using the SD-OCR biosensing arrangements of FIG. 1 and/or FIG. 3 and/or the arrangements described in International Patent Application PCT/US03/02349 to measure the depth-resolved information, e.g., at all interfaces simultaneously, and graph associated therewith which shows the outputs thereof;

FIG. 5 is an operational measurement diagram in accordance with an exemplary embodiment of the present invention using the SD-OCR biosensing arrangement of FIG. 1 and/or FIG. 3 and/or the arrangements described in International Patent Application PCT/US03/02349 which provides a multi-channel detection of the molecular interaction, and graph associated therewith which shows the outputs thereof;

FIG. 6a is an operational measurement in accordance with an exemplary embodiment of the present invention using the SD-OCR biosensing arrangement of FIG. 1 and/or FIG. 3 and/or the arrangements described in International Patent Application PCT/US03/02349 for monitoring a phase in the interference between reflected beams from top and bottom surfaces of a microfluidic device as a function of time;

FIG. 6b is an operational measurement in accordance with an exemplary embodiment of the present invention using the SD-OCR biosensing arrangement of FIG. 1 and/or FIG. 3 and/or the arrangements described in International Patent Application PCT/US03/02349 to performing the concentration monitoring procedure of FIG. 6a with the aid of a galvanometer beam scanner;

FIG. 7 is a graph illustrating exemplary Subsequent bBSA-streptavidin bindings measured by the exemplary SD-OCR biosensing arrangement according to the present invention;

FIG. 8a is a graph showing results of an exemplary controlled bBSA-streptavidin binding measurement illustrating an increase in a thickness at a bBSA-functionalized sensor surface;

FIG. 8b is a graph showing results of an exemplary controlled bBSA-streptavidin binding measurement which illustrates that no increase in the thickness was observed in a non-functionalized surface;

FIG. 9a is a graph showing an exemplary change of a cover slip thickness at a particular HF concentration in accordance with the present invention;

FIG. 9b is a graph showing an exemplary change of an etching rate at different HF concentrations in accordance with the present invention;

FIG. 10 is an exemplary graph of an image of a photosynthetic protein layer generated using the arrangement and method in accordance with the present invention; and

FIG. 11 is a flow diagram of an exemplary embodiment of the method according to the present invention.

Throughout the figures, the same reference numerals and characters, unless otherwise stated, are used to denote like features, elements, components or portions of the illustrated embodiments. Moreover, while the present invention will now be described in detail with reference to the figures, it is done so in connection with the illustrative embodiments.

### DETAILED DESCRIPTION OF EXEMPLARY EMBODIMENTS OF INVENTION

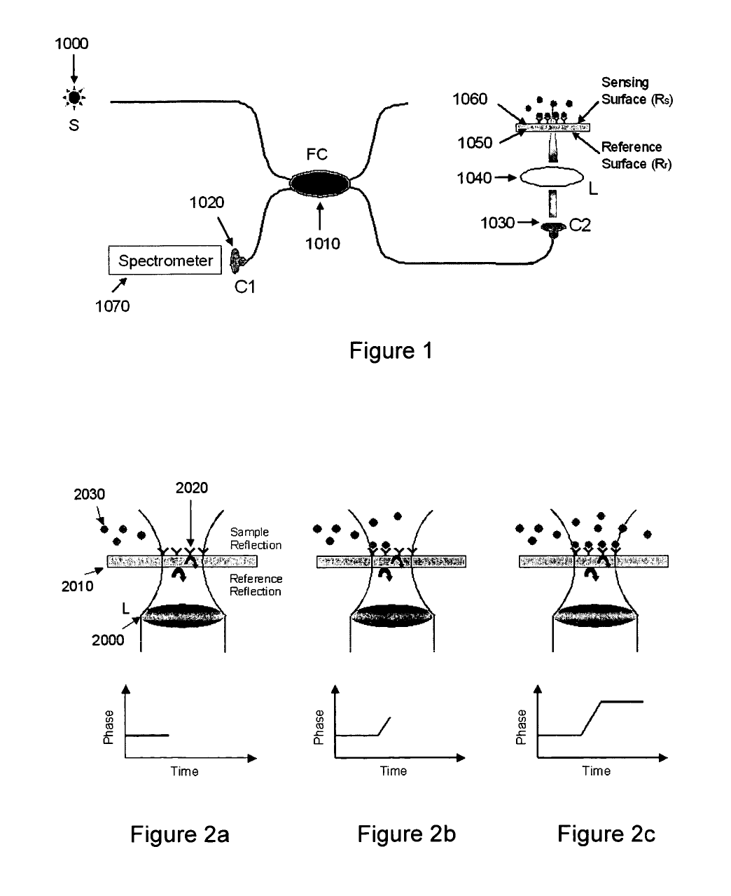

An exemplary embodiment of a fiber-based SD-OCR system according to the present invention is depicted as a diagram in FIG. 1. For example, as shown in FIG. 1, the system can include a broadband light source (1000) which may be configured to illuminate an interferometer (1010) such as a 2×2 fiber coupler, and the beam may be focused onto a sensing surface with a diffraction limited spot size. The sensing surface can be a protein/DNA chip or a part of a micro-fluidic device. The reflected beams from the interfaces of the sensing surface 1060 (and a glass 1050) can be re-coupled to the interferometer to produce an interference signal at the detection arm. At the spectrometer (1070), the signal related to the interference may be expressed as:
I(k)=2\sqrt{RᵣRₛ(z)}S(k)cos(2kΔp), (1)
where k is the wave number, z is the geometrical distance, and Rᵣ and Rₛ (z) represent the reference reflectivity and measurement reflectivity at depth z, respectively. S(k) is the power spectral density of the source, and Δp is the optical path length difference between the reference and measurement beams. A complex-valued depth information F(z) is obtained by a discrete Fourier transform of Equation (1) with respect to 2k, so the intensity and phase at depth z can be obtained as:

$$\begin{matrix}I ( z ) = {\vert F ( z ) \vert}^{2} , & ( 2 ) \\ ϕ ( z ) = {tan}^{- 1} [ \frac{Im ( F ( z ) )}{Re ( F ( z ) )} ] = 2 \frac{2 π}{{λ}_{0}} Δ p ( z ) , & ( 3 )\end{matrix}$$

where λ₀ is the center wavelength of the source. The depth-resolved intensity information in Equation (2) is used to locate a specific interference signal of interest, and the phase (or thickness) alteration at that signal is monitored in real-time for molecular recognition. Indeed, the spectrometer (1070) can measure power spectrum of the interference between the reference (bottom surface of a glass 1050) and the molecule-coupled sensing surface or slide (1060). The system also can include collimators (C1: 1020, C2: 1030), focusing lens (L: 1040) and spectrometer (1070).

For example, to perform an exemplary molecular absorption detection, exemplary probe molecules at the sensing surface can be immobilized or patterned via known protocols (as described in BIACORE Getting Started. 1998, Biacore AB). One of the ways to perform this can be by immersing the sensor surface in a high concentration solution of the probe molecules for several hours, and then rinse it with a Phosphate Buffered Saline (PBS) solution. In terms of patterning an array of probe molecules, this can be done by employing a micro-contact printing technique (as described in A. Bernard et al., “Microcontact printing of proteins,” Advanced Materials, 2000, Vol. 12, pp. 1067-1070), in which a polydimethylsiloxane (PDMS) stamp containing protein is brought into contact with the surface for physical absorption. After the sensor surface is activated with the probes, the analytes may be introduced to the sensing surface, as shown in FIGS. 2a-2c which illustrates an exemplary measurement of the molecular interaction using the exemplary system of FIG. 1. For example, probe molecules (2020) can be immobilized on the sensing surface (2010), and the molecules of interest (2030) can be introduced. As the analytes interact and bind to the probe molecules, the thickness at the sensor surface changes, and the reflection from the layer of bound molecules leads to a phase alteration in the interference signal being measured. In other words, as the molecules bind to the probe molecules, the phase change can be detected in real-time. This exemplary change is utilized to study the affinity of the analytes to the probe molecules and the kinetics associated with the interaction.

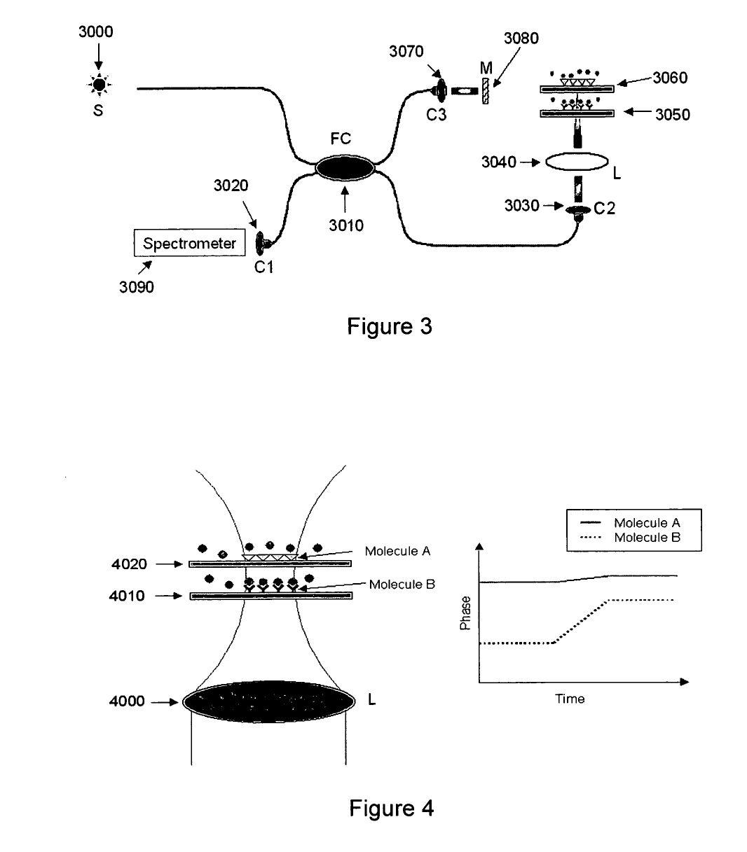

Exemplary embodiments of the system, arrangement and method according to the present invention can also provide a depth-resolved detection of molecular interactions, as shown in FIG. 3 which illustrates another exemplary embodiment of the SD-OCR arrangement which can perform a SD-OCR depth-resolved measurement of the molecular interaction. As shown in FIG. 3, the mirror (M: 3080) can be provided in the reference path, and the spectrometer (3090) may measure the power spectrum of the interference between the reflection from the reference mirror (M: 3080) and the reflections from the molecule-coupled glass slides (3050, 3060). In particular, this exemplary arrangement of FIG. 3 may further include a broadband light source (S: 3000), a 2×2 fiber coupler (FC: 3010), collimators (C1: 3020, C2: 3030, C3: 3070), a focusing lens (L: 3040), molecule-coupled glass slides (3050, 3060), and spectrometer (3090). For example, the interference can be measured between the reflected beam from the stationary mirror and the beams from the interfaces of the multilayer device is measured.

FIG. 4 illustrates an exemplary operational measurement in accordance with an exemplary embodiment of the present invention using the SD-OCR biosensing arrangement of FIG. 1 and/or FIG. 3 and/or the arrangements described in International Patent Application PCT/US03/02349 to measure the depth-resolved information, e.g., at most or all interfaces simultaneously, and a graph associated therewith which shows the outputs thereof. For example, the electro-magnetic radiation or light can be projected via one or more lenses L (4000), and molecule-coupled sensor surfaces (4010, 4020) shown in this figure can be activated with different molecules. An exemplary depth-resolved measurement based on these surfaces (4010, 4020) may indicate different affinities of molecules of interest with the immobilized molecules A and B. The intensity information can be used to identify each sensor surface (3050, 3060) shown in FIG. 3, and the phase of each such sensor surface can be monitored in real-time for analyzing kinetics of the same or similar analytes for the difference (probe) molecules, for example as shown in FIG. 4.

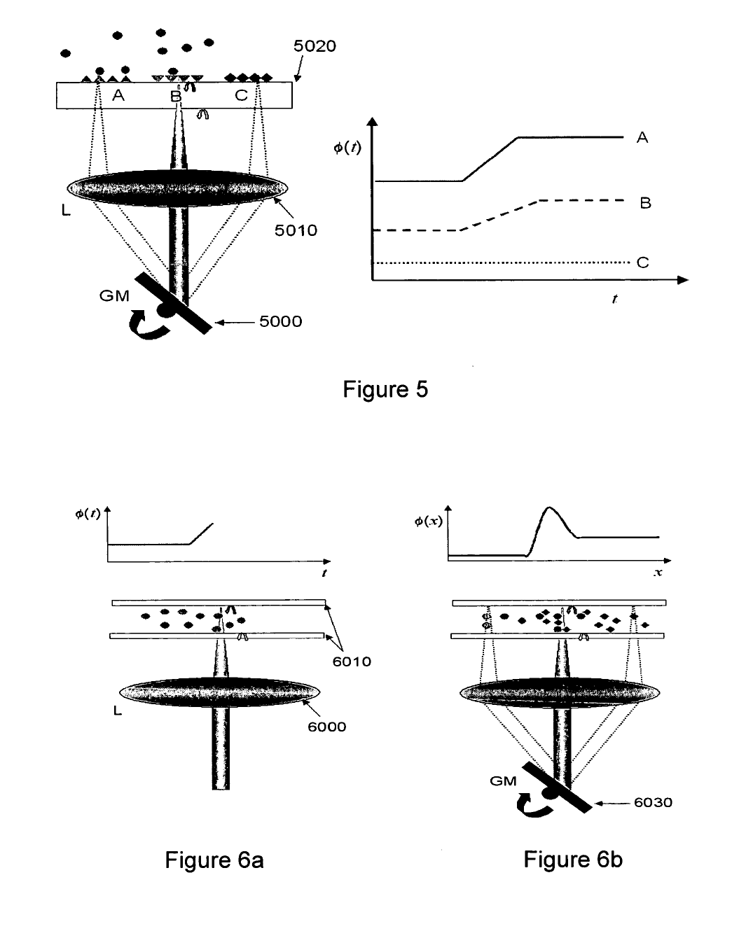

An exemplary embodiment of a high-throughput multi-channel detection of molecular bindings is possible via micro arrays of probe molecules as shown in the diagram and graph of FIG. 5. As shown in FIG. 5, which illustrates a galvanometer scanning mirror (GM: 5000), a focusing lens (L: 5010), and a multi-molecule coupled glass slide (5020), a sensor surface of the slide (5020) can be patterned with small features (1˜10 □m) of different probes, after which the free surface is saturated with inert proteins. As the molecules of interest, or analytes, are introduced, the probe beam scans across the sensing surface to monitor and measure molecular interactions in each of probe (or activation) sites in real-time. Since non-specific protein-protein binding (cross reactivity) is common to the entire sensor surface, it can be cancelled out by examining the entire sensor surface and by comparing the change in probe (or activated) regions with that of non-activated regions.

In addition to the detection of molecular absorption on a sensing surface, exemplary embodiments of the system, arrangement and method according to the present invention can also be used for measuring the amount (or concentration) of the free molecules in a fluidic channel. For example, the presence of the free molecules in a solution can change the effective refractive index in the channel, which may alter the phase in the interference between the reflected beams from the top and bottom surfaces of the channel. FIGS. 6a and 6b show operational illustrations of two exemplary depictions of such concepts, and include at least one focusing lens (L: 6000), a microfluidic device (6010), and a galvanometer beam scanner (GM: 6030). In FIG. 6a, the phase in the interference between the top and bottom walls of the fluidic channel is measured or monitored at one or more specific locations as a function of time, and the introduction of the molecules in the channel increases the phase measurement. Through an appropriate calibration, the exemplary embodiments of the present invention can be used to quantify the concentration level of the solution. FIG. 6b shows an operational diagram of how two different molecules diffuse in a fluidic channel. As provided in this drawing, the probe beam scans across the fluidic channel to measure the spatial phase distribution, as the molecules diffuse. As the molecules are flowing into the channel, e.g., between surfaces of the microfluidic device (6010), the phase change can be induced, which may indicate the change in the molecule concentration. For the diffusion measurement, the probe beam scans across the channel, and measure the spatial phase distribution caused by diffusion of these molecules. This measurement can be useful to quantify diffusion rate and binding affinity of label-free species for a given environment.

Supporting Data

I. Measurement of Biotin and Streptavidin Interaction

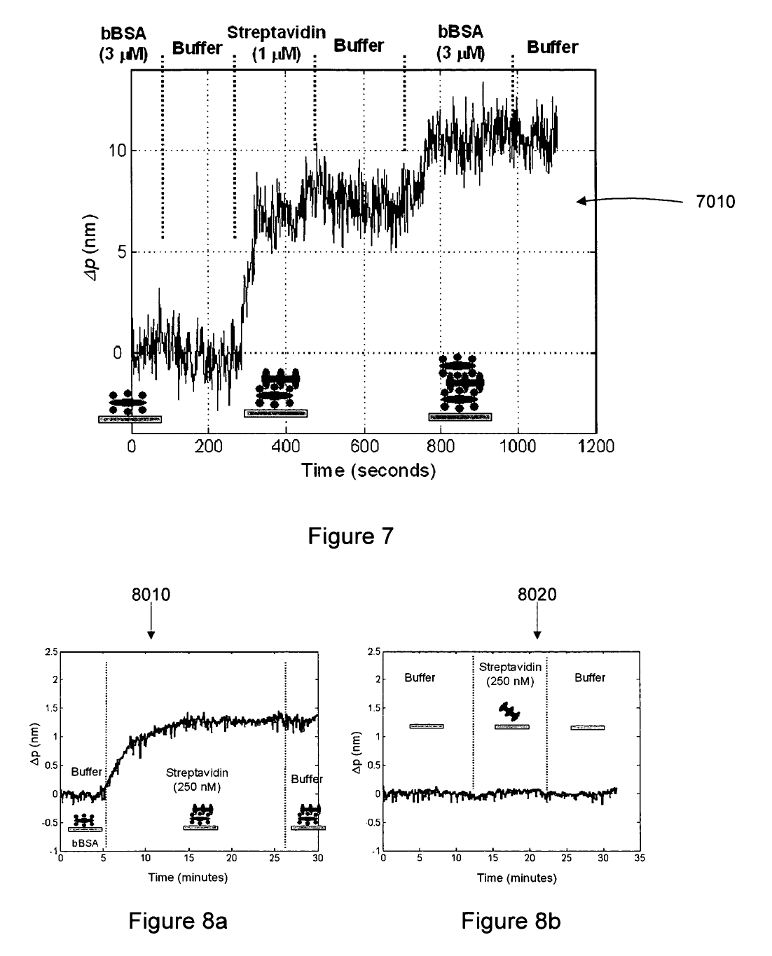

As a preliminary demonstration of the implementation of the exemplary embodiments of the present invention, the interaction between biotin and streptavidin at a sensor surface was measured as provided in FIG. 7, which shows a graph 7010 of exemplary Subsequent bBSA-streptavidin bindings measured by the exemplary SD-OCR biosensing arrangement according to the present invention. The interior channel of a micro-fluidic device was activated with biotinylated bovine serum albumin (bBSA), and several experiments were conducted to detect the subsequent bBSA-streptavidin bindings. Initially, introduction of PBS solution did not change the thickness at the sensing surface, but the noticeable change was observed after the streptavidin solution (1 μM) was injected into the exemplary device, due to the binding of the streptavidin to the immobilized bBSA layer. As shown in FIG. 7, the thickness remained constant after all the binding sites of bBSA were occupied by the streptavidin. The subsequent introduction of PBS solution did not change the thickness measurement. However, when the bBSA solution was flowed in again (3 μM), a further increase in the thickness was observed, which can be because the injection of streptavidin restored the ability to bind bBSA in the channel, as illustrated by bBSA-streptavidin multi-layer formation. The subsequent introduction of the buffer solution did not change the signal, but when switched back to bBSA solution, a further thickness increase was observed.

Control experiments with lower concentration of streptavidin solution (250 nM) were also conducted as provided in FIGS. 8a and 8b. For example, FIG. 8a shows a graph 8010 providing exemplary results of an exemplary controlled bBSA-streptavidin binding measurement illustrating an increase in an thickness at a bBSA-functionalized sensor surface. FIG. 8b shows a graph 8020 of exemplary results of the exemplary controlled bBSA-streptavidin binding measurement which illustrates that no increase in the thickness was observed in a non-functionalized surface. As shown in these drawings, the channel of a micro-fluidic device was functionalized with bBSA, and the streptavidin was introduced into the channel. The thickness increase was observed due to the binding of the streptavidin with slower rate, compared to a previous measurement. However, in the case of non-functionalized sensing surface, the thickness did not change, as shown in FIG. 8b, which demonstrates specific binding nature of streptavidin with biotin.

II. Detection of SiO₂ Etching

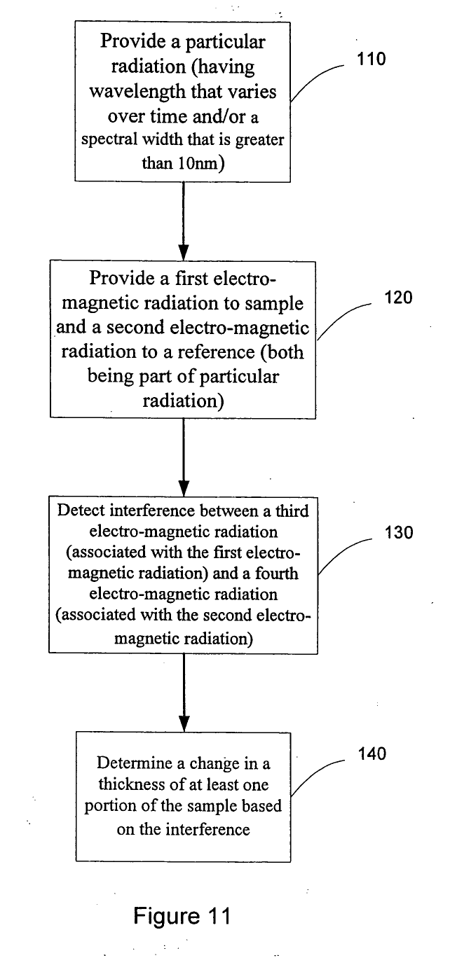

A flow diagram of the exemplary embodiment of the method according to the present invention is shown in FIG. 11. For example, a particular radiation having wavelength that varies over time and/or a spectral width that is greater than 10 nm can be provided by a source arrangement (step 110). Indeed, a first electro-magnetic radiation can be provided to sample and a second electro-magnetic radiation may be provided to a reference (both being part of particular radiation) as provided in step 120. Next, the interference between a third electro-magnetic radiation (associated with the first electro-magnetic radiation) and a fourth electro-magnetic radiation (associated with the second electro-magnetic radiation) can be detected in step 130. Further, a change in a thickness of at least one portion of the sample based on the interference can be determined in step 140.

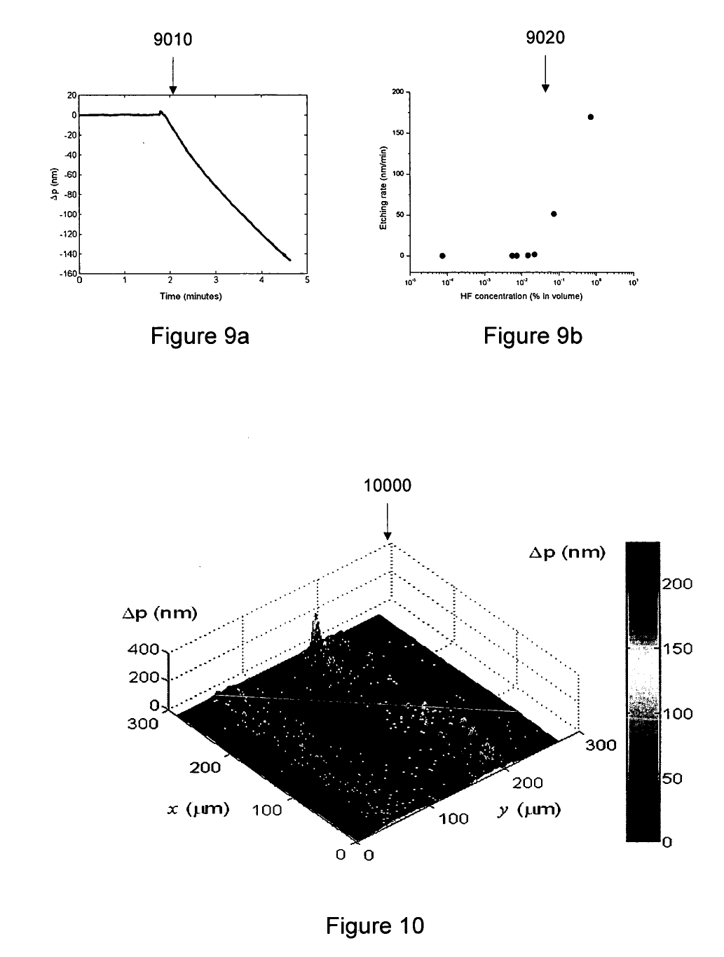

The exemplary embodiment of the method according to the present invention can be utilized to measure the number of silica molecules (SiO₂, MW: ˜60 Da) (as described in Handbook of Chemistry and Physics, 86 ed., 2005: CRC Press, p. 2544), etched by a diluted hydrofluoric acid (HF) solution. SiO₂ is a representative of small molecules, and its surface density is well known. In this example, a cover slip bottom culture dish (Mattek, Ashland, Mass.) was filled with de-ionized water, and the HF solution was injected into the dish to achieve desired concentrations. The probe beam at the cover slip surface had a diameter of ˜5 μm, and the changes of the effective thickness were monitored as a function of time. FIG. 9a shows a graph illustrating an exemplary change of a cover slip thickness at a particular HF concentration ˜0.07% in volume in accordance with the present invention. For this graph of FIG. 9a, the measured etching rate was ˜51 nm/min. A cover slip bottom culture dish was filled with de-ionized water, and the HF solution was injected into the dish to achieve desired concentrations (7×10⁻⁵˜0.7%). The change of the etching rate of the silica molecules was also measured, as varying the HF concentration, as shown in FIG. 9b which illustrates a graph of an exemplary large change of an etching rate at different HF concentrations in accordance with the present invention, e.g., when the HF concentration is over 0.05%.

III. Photosynthetic Protein Layer Imaging

The photo-synthetic proteins extracted from spinach were patterned onto a cover slip using a micro-stamp contact printing technique (as described in A. Bernard et al., “Microcontact printing of proteins,” Advanced Materials, 2000, Vol. 12, pp. 1067-1070), and the pattern of the proteins was imaged with the exemplary system, arrangement and method according to the present invention, as measuring the phase in the interference between reflections from top and bottom surfaces of the cover slip. FIG. 10 shows a graph 10000 of an image of a distribution of a photosynthetic protein layer generated using the arrangement and method in accordance with the present invention the surface. The thickness distribution across a cover slip was obtained by measuring phase in the interference between top and bottom surface of the cover slip. The photosynthetic protein layer was patterned by a micro-stamp contact printing technique. The result demonstrates the potential of the invention for imaging ultra thin organic layers or films.

There are several aspects of the exemplary embodiments of the system, arrangement and method according to the present invention in the implementation for chemical and biological species detection. For example, these exemplary embodiments can provide:
i. a label-free detection, e.g., a molecular recognition can be achieved without a specimen preparation such as fluorescence and radioactive labeling.
ii. the sensing area can be approximately as small as diffraction-limited size (˜1 micron), and the detection can be achieved with significantly reduced amount of molecules.
iii. the small size of the sensing area can permit monitoring multitudes of activated probe sites in parallel on two-dimensional disposable arrays.
iv. the exemplary measurement system and arrangement can be completely decoupled from microarrays or microfluidic devices, and thus may be deployed to any environments, and may not use the regeneration of the sensor surface.
v. the multi-layer depth-resolved molecular detection can be performed.
vi. the measurement can be achieved at microsecond temporal resolution, and the exemplary embodiment can be applied to fast kinetic procedures such as DNA denaturization.
vii. the exemplary embodiment can also be used to measure the concentration and diffusion of free molecules in micro-fluidic device.

The foregoing merely illustrates the principles of the invention. Various modifications and alterations to the described embodiments will be apparent to those skilled in the art in view of the teachings herein. Indeed, the arrangements, systems and methods according to the exemplary embodiments of the present invention can be used with any OCT system, OFDI system, SD-OCT system or other imaging systems, and for example with those described in International Patent Application PCT/US2004/029148, filed Sep. 8, 2004, U.S. patent application Ser. No. 11/266,779, filed Nov. 2, 2005, and U.S. patent application Ser. No. 10/501,276, filed Jul. 9, 2004, the disclosures of which are incorporated by reference herein in their entireties. It will thus be appreciated that those skilled in the art will be able to devise numerous systems, arrangements and methods which, although not explicitly shown or described herein, embody the principles of the invention and are thus within the spirit and scope of the present invention. In addition, to the extent that the prior art knowledge has not been explicitly incorporated by reference herein above, it is explicitly being incorporated herein in its entirety. All publications referenced herein above are incorporated herein by reference in their entireties.

What is claimed is:

1. A system comprising:
at least one first arrangement configured to provide a particular radiation which includes at least one first electro-magnetic radiation directed to at least one sample and at least one second electro-magnetic radiation directed to a reference;
at least one second arrangement configured to detect an interference between at least one third electro-magnetic radiation associated with the at least one first electro-magnetic radiation and at least one fourth electro-magnetic radiation associated with the at least one second electro-magnetic radiation; and
at least one third arrangement configured to determine a change in a thickness of at least one portion of the at least one sample based on the interference,
wherein the particular radiation has at least one of:
i. a wavelength provided by the at least one first arrangement that varies over time, or
ii. a spectral width that is greater than 10 nm.

2. The system according to claim 1, wherein the first and second radiations share a common path.

3. The system according to claim 1, wherein the at least one sample includes a plurality of samples, and wherein the change in the thickness of the at least one portion of each of the samples is determined simultaneously.

4. The system according to claim 1, wherein the change in the thickness of the at least one portion of the at least one samples is determined simultaneously at different locations at least one of along or perpendicular to a beam path of the at least one first electro-magnetic radiation.

5. The system according to claim, wherein the change in the thickness of the at least one portion of the at least one samples is determined simultaneously along different locations along a beam path of the at least one first electro-magnetic radiation.

6. The system according to claim 1, wherein the at least one first electro-magnetic radiation is scanned over a surface of the at least one sample at a plurality of locations thereon.

7. The system according to claim 1, wherein the at least one portion of the at least one sample is coated with particular molecules that are designed to associate with or dissociate from to further molecules.

8. The system according to claim 7, wherein the change of the thickness is associated with an association or a dissociation of the particular molecules.

9. The system according to claim 7, wherein the particular molecules have an affinity to bind to the further molecules that are different from the particular molecules.

10. The system according to claim 7, wherein the at least one portion includes a plurality of portions, wherein a first set of the particular molecules has an affinity to bind to a first portion of the plurality of portions, and the a second set of the particular molecules has an affinity to bind to a second portion of the plurality of portions, and wherein the first and second sets are different from one another.

11. The system according to claim 1, wherein the at least one sample has multiple layers therein.

12. The system according to claim 1, wherein the at least one sample is disposable.

13. The system according to claim 1, wherein the at least one sample is a micro-fluidic arrangement.

14. The system according to claim 1, wherein the change of the thickness of the at least one portion of the at least one sample is at least one of an optical thickness change, a physical thickness change or a refractive index change.

15. The system according to claim 14, wherein the thickness change is associated with a concentration of molecules at least one of on or in the at least one portion of the at least one sample.

16. The system according to claim 14, wherein the thickness change as a function of wavelength is associated with types of molecules at least one of on or in the at least one portion of the at least one sample.

17. The system according to claim 1, wherein the at least one first electro-magnetic radiation has a cross-section of a beam on or in the at least one portion of the at least one sample has a size that ie at least a diffraction-limited size.

18. The system according to claim 1, wherein the at least one third arrangement determines the thickness by:
i. transforming the interference into first data which is in a complex format,
ii. determining an absolute value associated with the first data to generate second data,
iii. identifying particular locations of the at least one portion as a function of the second data,
iv. determining a phase associated with the first data to generate third data, and
v. associating the change of the thickness with the third data.

19. The system according to claim 1, wherein the interference is Fourier transformed to generate the first data.

20. A method comprising:
providing a particular radiation which includes at least one first electro-magnetic radiation directed to at least one sample and at least one second electro-magnetic radiation directed to a reference;
detecting an interference between at least one third electro-magnetic radiation associated with the at least one first electro-magnetic radiation and at least one fourth electro-magnetic radiation associated with the at least one second electro-magnetic radiation; and
determining a change in a thickness of at least one portion of the at least one sample based on the interference,
wherein the particular radiation has at least one of:
i. a wavelength that varies over time, or
ii. a spectral width that is greater than 10 nm.
